(19) United States
(12) Patent Application Publication
(10) Pub. No.: US 2021/0361806 A1
(43) Pub. Date: Nov. 25, 2021

(54) SYSTEMS AND METHODS FOR DISINFECTING FOOD PACKAGING

(71) Applicant: Preservation Tech LLC, Hull, IA (US)

(72) Inventors: Kevin York, Joplin, MO (US); Jimmy Moler, Hull, IA (US); Sannel Patel, Hull, IA (US); Joe Rheingans, Hull, IA (US); Joel Beukelman, Hull, IA (US); Andy Shea, Hull, IA (US); Tyler Broich, Hull, IA (US); Tod Cunningham, Hull, IA (US); Christopher McCoy, Hull, IA (US)

(21) Appl. No.: 17/328,787

(22) Filed: May 24, 2021

Related U.S. Application Data
(60) Provisional application No. 63/029,017, filed on May 22, 2020.

(51) Int. Cl. A61L 2/20; A61L 2/18

(52) U.S. Cl. CPC A61L 2/202; A61L 2101/20; A61L 2/18

(57) ABSTRACT

Disclosed is a method for disinfecting packaging. In certain aspects, the method comprises placing the packaging in a contacting chamber and directly contacting the packaging with gaseous ozone for a time interval, wherein the time interval is sufficient to substantially reduce pathogens on the packaging. In certain embodiments, the time interval is from about 3 seconds to about 30 seconds. In certain aspects, the gaseous ozone is comprised of from about 6% to about 12% ozone and the time interval is sufficient to substantially reduce pathogens on the packaging.

### CROSS-REFERENCE TO RELATED APPLICATION(S)

This application claims priority to U.S. Provisional Application No. 63/029,017 filed May 22, 2020, and entitled “SYSTEMS AND METHODS FOR DISINFECTING FOOD PACKAGING which is hereby incorporated by reference in its entirety under 35 U.S.C. § 119(e).

### TECHNICAL FIELD

The disclosed technology relates generally to systems and method for disinfecting packaging.

### BACKGROUND

As meat processing facilities are continually looking for alternative technologies that balance cost versus result, further evaluations of different biocides having different molecular structures and different modes of action has become of significant interest. Alternative disinfection methods have become a part of any corporation's social responsibility plan to lower the environmental impact that harsh antimicrobials potential pose. In addition, the production of safe, healthy food requires that organizations pursue alternative means to lower the risk of food borne organisms why providing a safe environment for their employees. Furthermore, outside the food sector, the Covid-19 pandemic has increased the need for sanitation of packaging of all kinds. More tools are needed that provide an added layer of protection in lowering the risk of naturally occurring pathogenic organisms. Thus, there is a need in the art for improved biocides, that can be applied safely and economically.

### BRIEF SUMMARY

Disclosed herein is a method for disinfecting packaging. In certain aspects, the method comprises placing the packaging in a contacting chamber and directly contacting the packaging with gaseous ozone for a time interval, wherein the time interval is sufficient to substantially reduce pathogens on the packaging. In certain embodiments, the time interval is from about 3 seconds to about 30 seconds. In further embodiments, the time interval is from about 5 seconds to about 15 seconds. In yet further embodiments, the time interval is about 5 seconds.

In certain embodiments, the time interval is repeated upon repositioning of the packaging in the chamber. In exemplary implementations, the time interval is repeated three time for each side of the package.

According to certain embodiments, the gaseous ozone is introduced into the chamber at about 1 liters per minute. In certain implementations, the gaseous ozone is may include from about 6% to about 12% ozone. In further implementations, the gaseous ozone is from about 10% to about 12% ozone. In yet further implementations, the gaseous ozone is about 12% ozone.

Further disclosed herein is a method for disinfecting packaging by placing the packaging in a contacting chamber, the contacting chamber having a negative pressure; directly contacting the packaging with gaseous ozone for a time interval, wherein the gaseous ozone is comprised of from about 6% to about 12% ozone and wherein the time interval is sufficient to substantially reduce pathogens on the packaging. In certain implementations, the method may include treating the packaging with a liquid disinfectant, prior to introducing the packaging into the contacting chamber. In exemplary embodiments, the liquid disinfectant is peracetic acid PAA.

In further embodiments, a system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a system for disinfecting packaging. The system also includes a contacting chamber having a negative pressure, configured and arranged to apply gaseous ozone to packaging; an ozone generator, configured and arranged to provide gaseous ozone to the contacting chamber to directly contact the packaging with gaseous ozone; and an ozone monitor positioned outside of the contacting chamber; and a control module, configured and arranged to activate the ozone generator to deliver gaseous ozone to the contacting chamber for a time interval, wherein the gaseous ozone is comprised of from about 6% to about 12% ozone and wherein the time interval is sufficient to substantially reduce pathogens on the packaging Other embodiments of this aspect include corresponding computer systems, apparatus, and computer programs recorded on one or more computer storage devices, each configured to perform the actions of the methods.

In certain implementations, the time interval is from about 5 to about 15 seconds. Implementations of the described techniques may include hardware, a method or process, or computer software on a computer-accessible medium.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 a side view of a integrated oxygen concentrator and air compressor, according to certain embodiments.

FIG. 2 is an exemplary photographic image of the contacting chamber, according to certain embodiments.

FIG. 3 shows photographic images of treated and untreated packaging, according to certain embodiments.

FIG. 4 shows pathogen count data, according to certain embodiments.

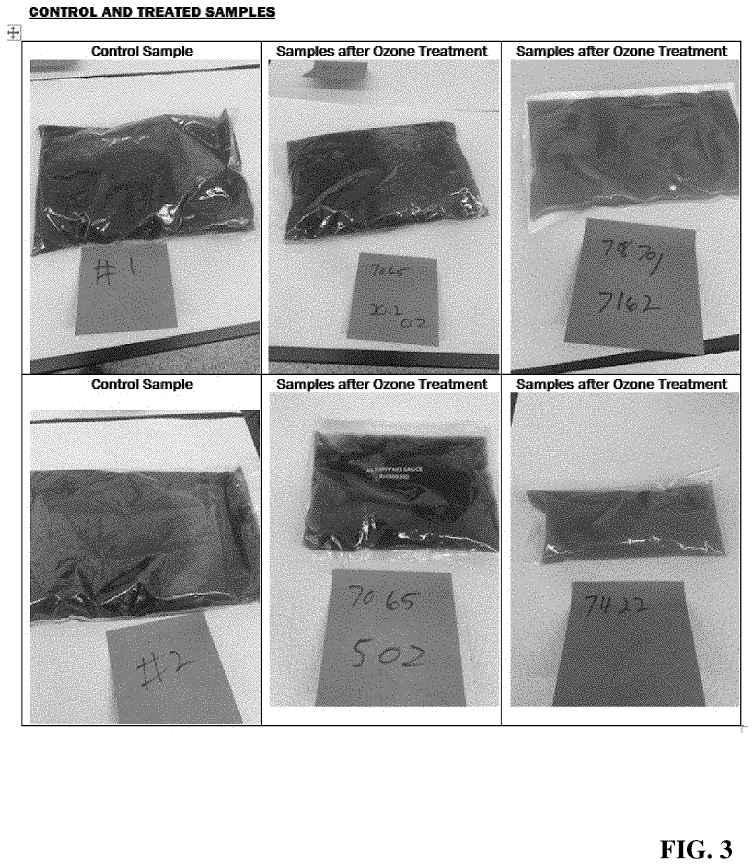

FIG. 3 shows pathogen count data, according to certain embodiments.

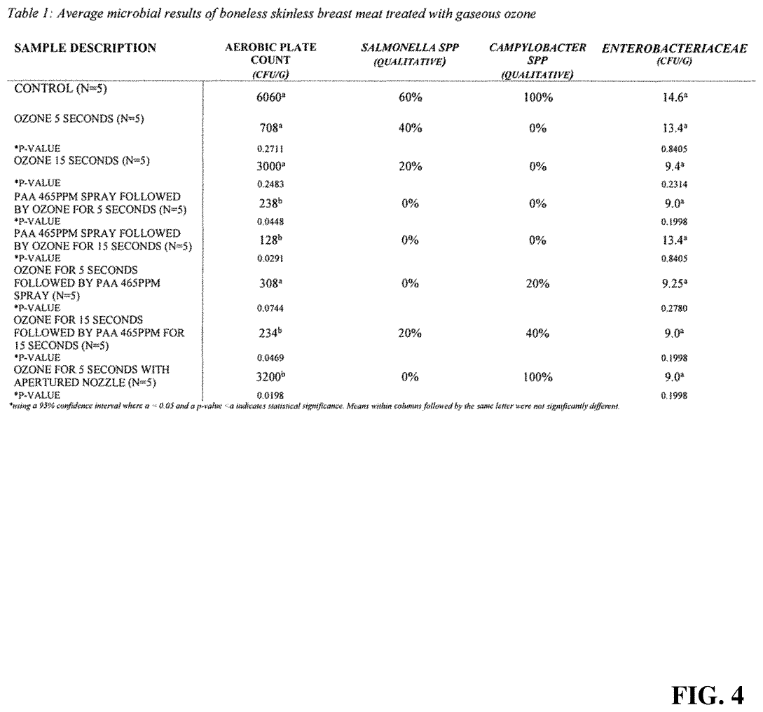

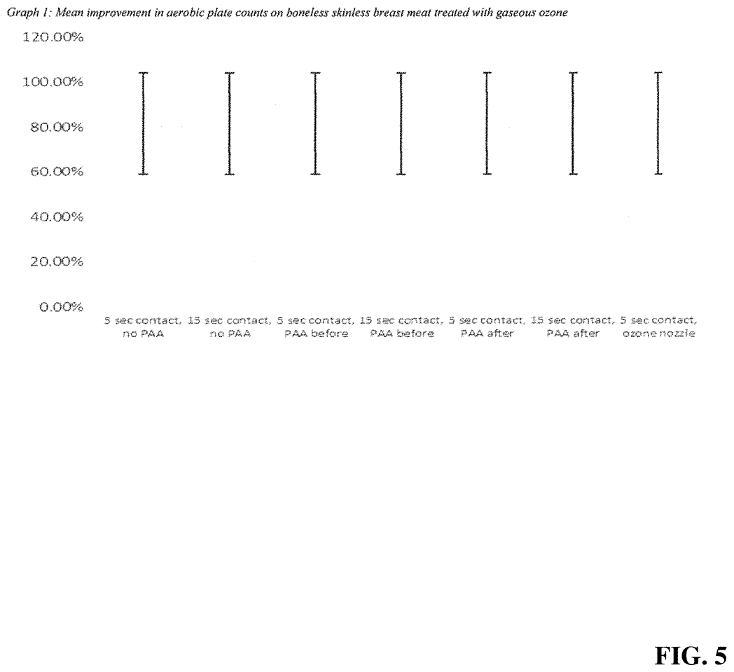

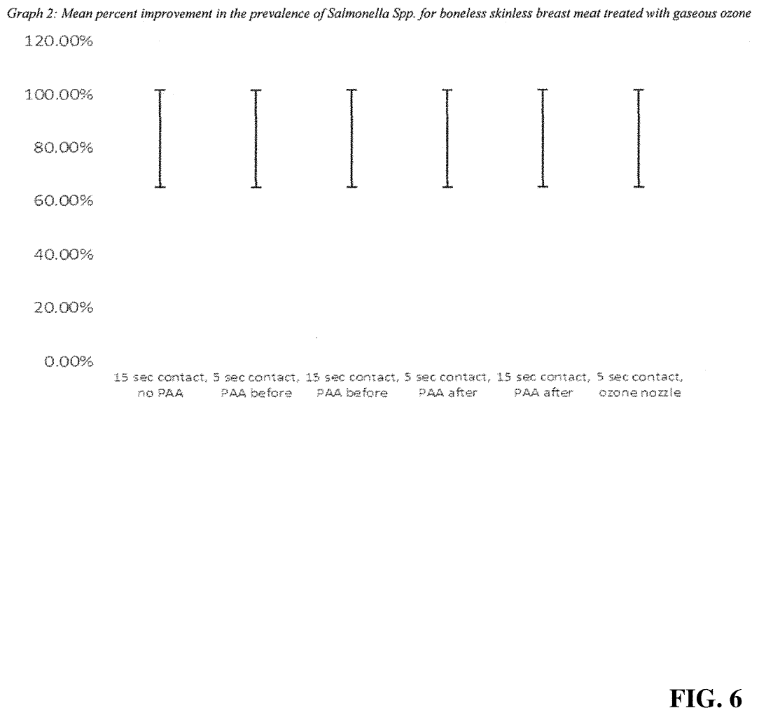

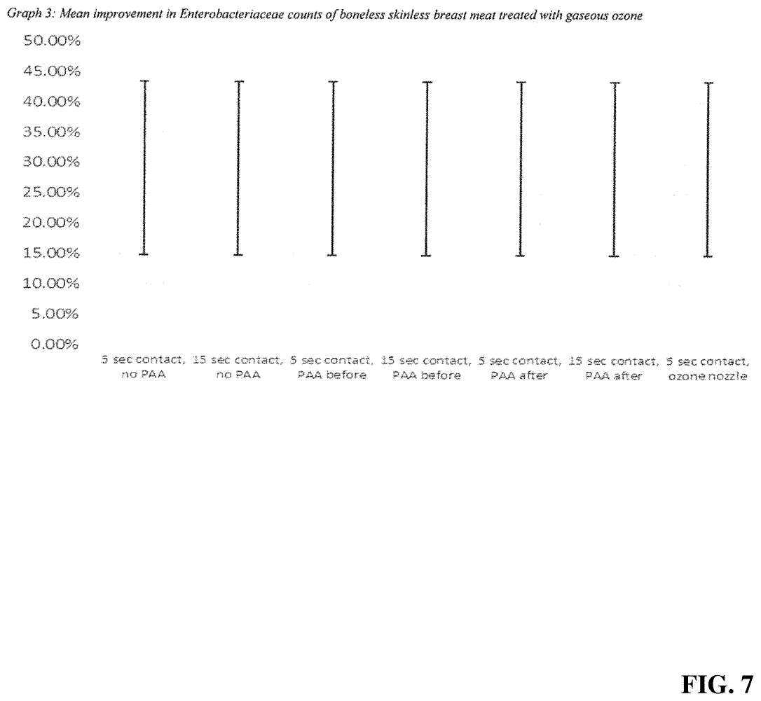

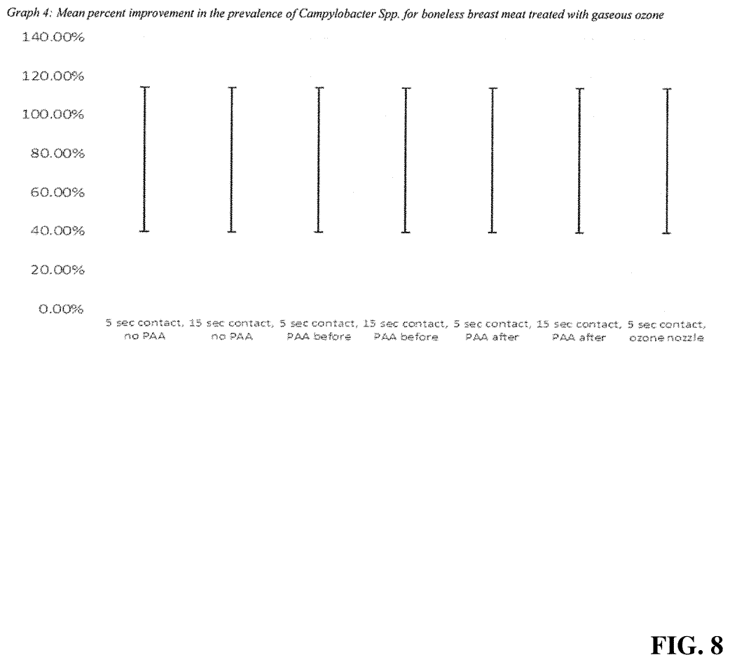

FIG. 4 shows pathogen count data, according to certain embodiments.

FIG. 5 shows pathogen count data, according to certain embodiments.

FIG. 6 shows pathogen count data, according to certain embodiments.

FIG. 7 shows pathogen count data, according to certain embodiments.

FIG. 8 shows pathogen count data, according to certain embodiments.

### DETAILED DESCRIPTION

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, unless expressly stated otherwise, “inside” and “inner” means closer to the wrapped product and “outer” means toward an exterior surface of the packaging.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is substantially free of particles would either completely lack particles, or so nearly completely lack particles that the effect would be the same as if it completely lacked particles. In other words, a composition that is substantially free of an ingredient or element may still actually contain such item as long as there is no measurable effect thereof.

Ozone, a gas that is a triatomic form of oxygen, has been used for years in applications such as treatment of municipal water and bottled water. Ozone has enjoyed a long history of use and is known as a broad-spectrum biocide against viruses, bacteria, biofilms, fungi and protozoa—none of which can build up a resistive tolerance to ozone because ozone disinfects by oxidation processes. Ozone does not act as a systemic poison to microorganisms, but rather, destroys them by oxidation consequently making it impossible for a microorganism to build up any resistance to oxidation.

Disclosed herein is a method for disinfecting a package (in exemplary embodiments, packaged food) by placing the package in a contacting chamber and directly contacting the package with gaseous ozone for a time interval, wherein the time interval is sufficient to substantially reduce any pathogens on the package.

In certain aspects, time interval is from about 3 seconds to about 30 seconds. In further aspects, the time interval is from about 5 seconds to about 15 seconds. In yet further aspects, the time interval is about 5 seconds. In further embodiments, the time interval is divided into shorter time intervals and the package is reoriented between the shorter time intervals to ensure all surfaced of the package are exposed to the ozone treatment. In exemplary implementations, package is gassed with ozone for five seconds on a first side and then flipped over then treated for 5 seconds on a second side. This procedure can be repeated multiple times. In exemplary implementations, this procedure is repeated three times, resulting in a cumulative exposure of 15 seconds per package side. As will be appreciated by one skilled in the art, other intervals are possible.

According to certain embodiments, the gaseous ozone is introduced into the chamber at about 10 liters per minute. In further aspects, the gaseous ozone is comprised of from about 6% to about 12% ozone.

According to certain embodiments, the pressure in the contacting chamber is lower than ambient pressure e.g., there is a negative pressure in the contacting chamber.

According to further embodiments, the method further comprises treating the packaging with a liquid disinfectant, prior to introducing the packaging into the contacting chamber. In exemplary aspects of these embodiments, the liquid disinfectant is peracetic acid (PAA).

As will be appreciated by a person skilled in the art, the disclosed method may be utilized with food packaging in various forms. In certain aspects, the packaged food product contains poultry, beef, lamb, or pork. In further aspects, the packaging contains a meat product that is a combination of the foregoing. According to further aspects, the packaging contains a food condiment or sauce (e.g., salad dressing).

Further disclosed herein is a system disinfecting a packaged food item

The disclosed methods can be used to treat packages containing meat products in a variety of forms, including meat cuts and ground meat (for example, ground beef). Examples of meat cuts include primal cuts, subprimal cuts and retail cuts. Primal cuts include beef loins, pork loins, beef ribs, pork hams, and beef rounds. Subprimal cuts include beef strips, beef rib eyes, beef top sirloins, pork shoulder butts, pork center cut loins, pork sirloins, and beef bottom round flats. Retail cuts include sirloin steaks, stew meat, cube steaks, country style ribs, pork chops, blade steaks, cutlets, poultry thighs, poultry breasts, and poultry tenders.

In further embodiments, a system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a system for disinfecting packaging. The system also includes a contacting chamber having a negative pressure, configured and arranged to apply gaseous ozone to packaging; an ozone generator, configured and arranged to provide gaseous ozone to the contacting chamber to directly contact the packaging with gaseous ozone; and an ozone monitor positioned outside of the contacting chamber; and a control module, configured and arranged to activate the ozone generator to deliver gaseous ozone to the contacting chamber for a time interval, wherein the gaseous ozone is comprised of from about 6% to about 12% ozone and wherein the time interval is sufficient to substantially reduce pathogens on the packaging Other embodiments of this aspect include corresponding computer systems, apparatus, and computer programs recorded on one or more computer storage devices, each configured to perform the actions of the methods.

In certain implementations, the time interval is from about 5 to about 15 seconds. Implementations of the described techniques may include hardware, a method or process, or computer software on a computer-accessible medium.

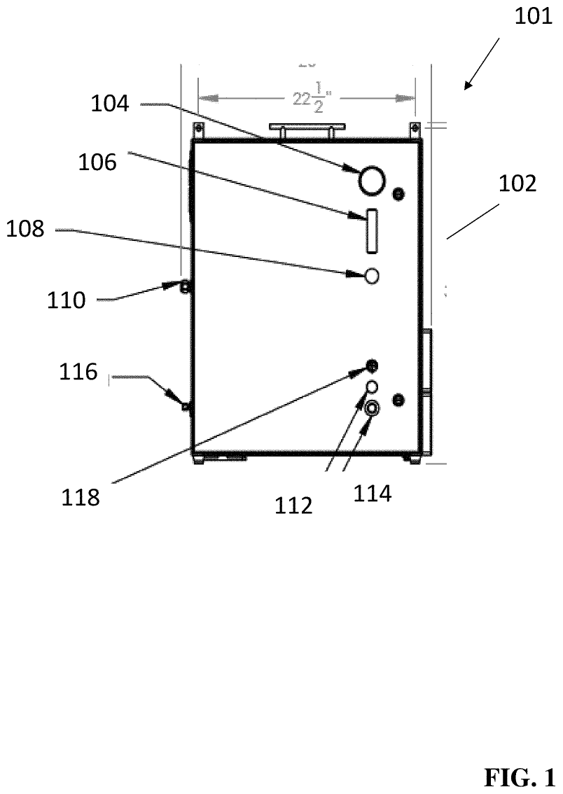

In certain implementations, the ozone generator is integrated oxygen concentrator and air compressor unit. In exemplary implementations, best shown in FIG. 1 the ozone generator 101, comprises housing 102, and oxygen pressure gauge 104, an oxygen flow meter 106, a needle valve 108 a power cable grip 110, an ozone generation lamp 112, an generator power level knob 114, ozone out port 116 and a power switch 118.

### EXPERIMENTAL EXAMPLES

The following examples are put forth so as to provide those of ordinary skill in the art with a complete disclosure and description of how the articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Materials & Methods

Equipment List:

1. Ozone Generator—TG—20 (FIG. 1)

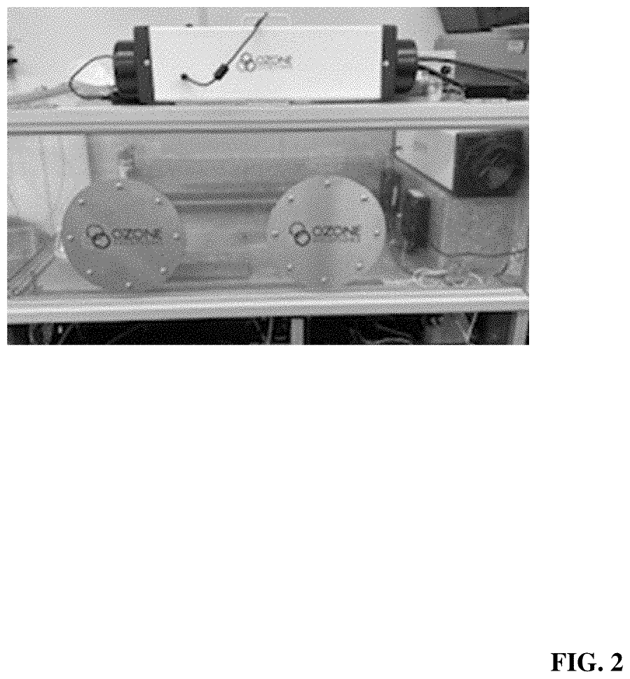

2. Handheld Ozone Monitor D16 monitor (0-2 ppm sensor) (FIG. 2)

3. High Level Gas Ozone Analyzer (UV-106H) (FIG. 3)

4. Ozone Chamber (FIG. 4)

Packages sent to location; Ozone Solutions Inc. 451 Black Forest Road, Hull, Iowa, 51239

Packages labeled as shown in FIG. 5. Sizes of packages were as follows:
5.5″×4.5″
6″×9″
6.5″×3″
7″×4.5″

Ozone Generation and Application
Ozone was be generated using an ozone generator at 1 LPM—12% by weight and applied to the package.
The package was gassed with ozone for five seconds on the top and then flipped over
The package was then gassed on the bottom for five seconds.
This was repeated 3 times.
The package was placed on the numbered grid and kept in a cooler for 48 hours and observations were recorded at 0, 24, and 48 hours.

### Data Analyses and Results

Once the packages were removed from the ozone chamber, no change or leakages were seen. The packages were observation during the 48 hours in 8-hour intervals, no reaction or change was noticed on any package, as shown in FIG. 6.

### Conclusion

No change was noticed on the packages after ozone treatment. Packages showed no sign of leakage at high ozone concentration for fifteen seconds.

### Example 2—Microbial Killing on Meat

Ozone Treatment of Meat

### Materials & Methods

Equipment List:
40 g/hr. Mobile zone Ozone Generator with Injection Pump Skid
Hand Held Ozone Monitor
Poultry parts Conveyor as supplied by the plant

Microbial analysis of raw poultry product via Aerobic Plate Count (APC) Petrifilm,

Ozone Generation:
The Ozone Generator is powered and the settings on the mobile zone screen should be set to ozone production to 0%.
Once the Start Button is pressed. The Compressor and oxygen concentrator will start to run.
Once the Oxygen runs for 120 seconds, then change the ozone production setting to 100%. Ozone will be then be generated.
Ozone will be set to 0% output between samples.
Monitor ambient ozone levels at intervals as measured by the ambient ozone monitor to determine that the ambient ozone levels are within OSHA limits.

Additional Measurements for Experiment: PAA Levels

Ozone Testing Procedure: A conveyor was tented with plastic sheeting to contain the ozone gas. A fan and destruct unit was connected to keep negative pressure in the chamber and prevent the ozone gas from escaping into the work area. The chicken breast was transferred from the debone line before bone inspection and placed on the conveyor. The conveyor speed was set to vary the exposure time from 5 seconds and 15 seconds. PAA was applied before ozone on one sample set and after ozone on an additional sample set. Sample size was 6 breasts, for each contact time.
Each breast was placed in a sterile rinse bag,
400 ml of BPW was added, shaken for one minute,
100 ml of rinsate was poured into the sample cup,
Sample cup was placed in cooler on top of cardboard and ice packs.

Experimental Design:

To determine the effect of ozone gas on poultry breast meat, the follow 8 treatment groups were tested (Table A),
Group 1—Control group with no treatment to assess microbial counts
Group 2—Conveyor speed set to 5 second exposure, no PAA
Group 3—Conveyor speed set to 15 second exposure, No PAA
Group 4—Conveyor speed set to 5 second exposure, PAA before Ozone
Group 5—Conveyor speed set to 15 second exposure, PAA before Ozone
Group 6—Conveyor speed set to 5 second exposure, PAA after Ozone
Group 7—Conveyor speed set to 15 second exposure. PAA after Ozone
Group 8—Conveyor speed set to 5 second exposure, no PAA, with nozzle

TABLE A
Control and Treatment Groups

| | | | | Sample |
|---|---|---|---|---|
| | | | | Num- |
| Groups | Name of Group | Treatment | Time of treatment | bers |
| Group 1 | Treatment- A | None | None | A1-A6 |
| | Control | | | |
| Group 2 | Treatment- B | No PAA | 5 seconds dwell time | B1-B6 |
| Group 3 | Treatment- C | No PAA | 15 seconds dwell time | C1-C6 |
| Group 4 | Treatment-D | PAA Before | 5 seconds dwell time | D1-D6 |
| Group 5 | Treatment-E | PAA Before | 15 seconds dwell time | E1-E6 |
| Group 6 | Treatment-F | PAA After | 5 seconds dwell time | F1-F6 |
| Group 7 | Treatment-G | PAA After | 15 seconds dwell time | G1-G6 |
| Group 8 | Treatment-H | No PAA | 5 seconds dwell time | H1-H6 |
| | | | With nozzles | |

### Statistical Analysis

Statistical analysis is performed using SAS. A Student's T-test will be utilized to separate the means for all quantitative data in determination of statistical significance.

### Results & Discussion

| | | | Log | | |
|---|---|---|---|---|---|
| | | | Reduc- | | |
| | | | tion | Average | |
| Treat- | | APC | from | APC | |
| ment | | Log10 | Control | Counts | |
| A | Control - No Treatment | 3.78 | | 6060 | |
| B | 5 sec contact, no PAA | 2.85 | 0.93 | 708 | n = 5 |
| C | 15 sec contact, no PAA | 3.48 | 0.31 | 3000 | |
| D | 5 sec contact, PAA before | 2.38 | 1.41 | 238 | |
| E | 15 sec contact, PAA before | 2.11 | 1.68 | 128 | |
| F | 5 sec contact, PAA after | 2.49 | 1.29 | 308 | |
| G | 15 sec contact, PAA after | 2.37 | 1.41 | 234 | |
| H | 5 sec contact, ozone nozzle | 3.51 | 0.28 | 3200 | |

Each sample consisted of 6 breasts weighing approximately 4 pounds. Samples were rinsed in BPW, put on ice and shipped overnight to a third-party lab. PAA control samples were not run as part of this experiment.

Ozone nozzle for the treatment H was the same application as treatment B, except for the nozzles.

In this trial, the microbiological quality of Aerobic Plate Count ‘APC’, Enterobacteriaceae ‘EB’ and Salmonella Spp. of poultry parts was treated with gaseous ozone and compared to microbiological levels of poultry parts prior to any antimicrobial treatments. Seven treatment groups were included in this trial and include:
Ozone gas @ 5 seconds,
Ozone gas @ 15 seconds
PAA spray followed by ozone gas @ 5 seconds
PAA spray followed by ozone gas @ 15 seconds
Ozone gas followed by PAA spray for 5 seconds
Ozone gas followed by PAA spray for 15 seconds
Ozone gas forced through a small aperture fan nozzle for 5 seconds.

### Results and Discussion

The samples were sent to the facility laboratory for microbial analysis of Aerobic Plate

Counts ‘APC’, Salmonella Spp., Campylobacter Spp., and Enterobacteriaceae ‘EB’. Quantitative results were transformed into colony forming units per gram (CFU/g). Microbial analysis results for each part were then averaged among control and treated groups (Table 1/FIG. 4). A Students T-Test was utilized to separate the means for the quantitative data to determine the statistical significance of the microbial results. As summarized in Table 1, boneless skinless breast meat receiving no antimicrobial treatment resulted in an Aerobic Plate Count of 6060 CFU/g, a 60% prevalence rate of Salmonella Spp., 100% prevalence rate of Campylobacter Spp., and a Enterobacteriaceae level of 14.6 CFU/g. When an antimicrobial treatment of gaseous ozone was applied for approximately 5 seconds to both sides of the substrate, the result was an APC level of 708 CFU/g or an 88.3% reduction in counts while the quantitative amount of Enterobacteriaceae was reduced to 13.4 CFU/g which represents an 8.2% reduction from control. Salmonella and Campylobacter organisms were reduced to levels of 40% and none detectable respectively. When the substrate was exposed to a 15 second treatment of gaseous ozone, the corresponding APC and EB levels were reduced to 3000 CFU/g and 9.4 CFU/g respectively or a 50.5% and 35.6% reduction while Salmonella was reduced to an incidence level of 20% and no Campylobacter was detected. When the addition of a per acetic acid spray was introduced on the top surface of the product prior to a 5 second application of ozone gas, the levels of APC and EB were reduced to 238 CFU/g and 9 CFU/g respectively. This represents a 96% and 38.3% reduction from control. There were no findings of either Salmonella or Campylobacter on the product tested for this treatment group. When the peracetic acid was applied prior to a 15 second ozone gas treatment, the APC and EB counts were reduced to 128 CFU/g and 13.4 CFU/g respectively or by 97.8% and 8.2% whereas there were no findings of either Salmonella or Campylobacter on the product tested. When the peracetic acid was applied after a 5 second ozone gas treatment, the corresponding levels of APC and EB were 308 CFU/g and 9.25 CFU/g which is a 95% and 36.6% improvement from control while no presence of Campylobacter was detected and Salmonella prevalence was reduced to 20%. The effect of expanding the dwell time of the ozone gas to 15 seconds coupled by a spray of peracetic acid resulted in a APC and EB counts of 234 CFU/g and 9 CFU/g accordingly. This translates into a reduction in APC of 96.1% and a 38.3% reduction in Enterobacteriaceae. The incidence of Salmonella and Campylobacter in this treatment group was 40% and 20% respectively. The inclusion of a specifically designed nozzle to isolate the ozone gas as a direct product contact on the surface of the product did reduce Aerobic Plate Count to 3200 CFU/g and Enterobacteriaceae counts to 9 CFU/g. Comparatively this represents a 47.2% and 38.3% improvement in APC and EB levels when compared to non-treatment. There was no corresponding reduction in Campylobacter;

In conclusion, gaseous ozone as a single point intervention was able to reduce bacteriological counts on further processed boneless skinless breast meat. When a spray application of peracetic acid was introduced either before or after the ozone treatment, the reduced microbiological counts were statistically significant. From a pathogen performance standpoint, ozone gas did lower pathogen levels on the product for all treatment groups and combinations except for the nozzle treatment which failed to reduce Campylobacter prevalence. When time was increased for the application of ozone gas on the surface of the product, the microbiological results failed to decrease in linear fashion. It can be determined that a 5 second application time would be sufficient to maximize the efficiency of microbial reductions with ozone gas. When comparing the nozzle application of ozone gas to an ambient distribution of ozone gas, the nozzle set-up failed to significantly demonstrate better results than the ambient distribution method.

Although the disclosure has been described with reference to preferred embodiments, persons skilled in the art will recognize that changes may be made in form and detail without departing from the spirit and scope of the disclosed apparatus, systems and methods.

What is claimed is:

1. A method for disinfecting packaging comprising:
a. placing the packaging in a contacting chamber;
b. directly contacting the packaging with gaseous ozone for a time interval, wherein the time interval is sufficient to substantially reduce pathogens on the packaging.

2. The method of claim 1, wherein the time interval is from about 3 seconds to about 30 seconds.

3. The method of claim 2, wherein the time interval is from about 5 seconds to about 15 seconds.

4. The method of claim 3, wherein the time interval is about 5 seconds.

5. The method of claim 4, wherein is the time interval is repeated upon repositioning of the packaging in the chamber.

6. The method of claim 5, wherein the time interval is repeated three time for each side of the package.

7. The method of claim 1 wherein the gaseous ozone is introduced into the chamber at about 1 liters per minute.

8. The method of claim 7, wherein the gaseous ozone is comprised of from about 6% to about 12% ozone.

9. The method of claim 8, wherein the gaseous ozone is comprised of from about 10% to about 12% ozone.

10. The method of claim 9, wherein the gaseous ozone is comprised of about 12% ozone.

11. A method for disinfecting packaging comprising:
a. placing the packaging in a contacting chamber, the contacting chamber having a negative pressure; and
b. directly contacting the packaging with gaseous ozone for a time interval, wherein the gaseous ozone is comprised of from about 6% to about 12% ozone and wherein the time interval is sufficient to substantially reduce pathogens on the packaging.

12. The method of claim 11, wherein the time interval is from about 3 seconds to about 30 seconds.

13. The method of claim 12, wherein the time interval is from about 5 seconds to about 15 seconds.

14. The method of claim 13, wherein the time interval is about 5 seconds.

15. The method of claim 11, further comprising treating the packaging with a liquid disinfectant, prior to introducing the packaging into the contacting chamber.

16. The method of claim 15, wherein the liquid disinfectant is PAA.

17. The method of claim 11, wherein is the time interval is repeated upon repositioning of the packaging in the chamber.

18. The method of claim 17, wherein the time interval is repeated three time for each side of the package.

19. A system for disinfecting packaging comprising:
a. a contacting chamber having a negative pressure, configured and arranged to apply gaseous ozone to packaging;
b. an ozone generator, configured and arranged to provide gaseous ozone to the contacting chamber and directly contact the packaging with gaseous ozone;
c. an ozone monitor positioned outside of the contacting chamber; and
d. a control module configured and arranged to activate the ozone generator to deliver gaseous ozone to the contacting chamber for a time interval, wherein the gaseous ozone is comprised of from about 6% to about 12% ozone and wherein the time interval is sufficient to substantially reduce pathogens on the packaging.

20. The system of claim 19, wherein the time interval is from about 5 to about 15 seconds.
